(19) United States
(12) Patent Application Publication
(10) Pub. No.: US 2012/0330216 A1
(43) Pub. Date: Dec. 27, 2012

(54) METHODS AND APPARATUS FOR SKIN REJUVENATION

(76) Inventor: Saad Ibrahim Almohizea, Riyadh (SA)

(21) Appl. No.: 13/168,815

(22) Filed: Jun. 24, 2011

(51) Int. Cl. A61M 35/00

(52) U.S. Cl. 604/20; 604/310; 604/290

(57) ABSTRACT

Apparatus and methods are provided for the purpose of applying topical treatment on the skin in a standardized and fractionated way. In one aspect of the inventive subject matter, a device includes a trigger activating a pump, which is continuous with a passageway that drains fluid from a reservoir to nozzle. The trigger is operatively coupled to the pump. The pump forces a predetermined volume of fluid to leave the nozzle at a predetermined distance from the skin.

### FIELD OF THE INVENTION

The present invention relates to the cosmetic treatment of skin by the use of compounds that exfoliate the skin in a controlled manner to enhance the appearance of the skin.

### BACKGROUND

Chemical peels are used to exfoliate the skin in a controlled manner to rejuvenation and treat various skin diseases. They include, but are not limited to trichloroacetic acid, phenol, glycolic and salicylic acid peels. After prepping the skin with acetone or alcohol, the peel of choice is applied to the skin with the use of a gauzes or cotton-tip applicators in one or more coats until the end point is reached. The patient goes home anticipating the healing phase to last anywhere from days to weeks. During this healing phase, skin usually starts to peel off, and new skin formation takes place as the redness and oozing fades away. This phase is known as downtime period. As the dead skin sloughs off, the new skin is usually smoother and less wrinkled than the old skin with a better texture and an even skin color.

The wounds created by these compounds stimulate the skin to synthesize collagen and induce peeling of the epidermis leading to skin regeneration. The result is a healthier skin with improved texture, smoothening of wrinkles, tightening of skin and amelioration of pigmentary changes. Therefore chemical peels are used to treat signs of aging and photodamage such as wrinkles and laxity. It has also been used for various diseases. to name a few, precancerous lesions such as actinic keratosis, Pigmentary problems such as melasma, dyschromias and freckles, hormonal problems such as acne and epidermal growths such as wart and milia can all be treated with chemical peels.

Aging skin is characterized by wrinkling, rough texture and uneven pigmentation. These characteristics are associated with decreased elastin, collagen, epidermal atrophy, cellular atypia, and dysplasia. These changes presumably result from DNA mutation and other cellular and protein damage. The consequence is abnormal collagen, Elastin, and ground substance breakdown. Cumulative sun exposure and smoking are some of the causes of photodamaged skin.

Depending on the depth of these peels, they have been classified into superficial, medium and deep peels. When the peel extends to only the epidermis they are known as superficial peels. Whereas medium depth peels extend into upper reticular dermis and deep peels into mid reticular dermis. The type of the peel, its concentration, and the duration of treatment determine the depth of the peel.

As with any medical procedure, chemical peels may be associated with complications. This is especially true with deep peels with all of the epidermis and a huge portion of the dermis damaged leaving the skin vulnerable during the healing phase. A common complication seen in phenolic deep peels is post inflammatory hypopigmentation. Scarring, post inflammatory hyperpigmentation, persistent erythema and delayed healing are other examples. Furthermore, since the deep peels produce deep oozing wounds, there is a higher rate for infection and a long downtime, the patient may wait weeks to months before he or she is completely healed. These complications are thoroughly discussed in many studies. In one reference TCA above 45% has been found to be unreliable and dangerous with a high incidence of scarring and postoperative complications (Chemical peels, Monheit G D, Skin Therapy Lett. 2004 Feb; 9(2):6-11.)

One of the major drawbacks of peels is the unpredictability of results and complications. This arises mainly because up until now, they are administered with the use of gauzes or cotton tip applicators with many application techniques. The force by which the applicator is pushed onto the skin and the volume of peel delivered to the skin varies greatly from one patient to another and even in the same patient since the applicator is usually wetted again before completing the treatment. With each stroke the peel's volume is highest at the beginning and dries off as the gauze is swept across the skin. This is even more complicated by the curvy nature of the skin that prevents standardizing the treatment. These drawbacks were emphasized in a paper published by Stone PA. (Modified phenol chemical face peels: recognizing the role of application technique. Stone P A, Lefer L G, Facial Plast Surg Clin North Am. 2001 Aug; 9(3):351-76.)

U.S. Pat. No. 5,562,643 teaches a device that propels fluid in a stream confined within a chamber that extends from a nozzle. The reference teaches placement of the device onto the skin to confine the fluid, and uses a vacuum source to ensure that the fluid remains on the skin. According to the teachings of the patent, the device is only suitable to a small area of skin such as small pitted skin scars. Giving a stream of peeling solution to a full face is not only dangerous, but would lead to unpredictable depth of peeling and complications. Furthermore, it is desirable to watch for skin reaction before giving another coat of peel, and that cannot realistically be done using such apparatus.

These and all other extrinsic materials discussed herein are incorporated by reference in their entirety. Where a definition or use of a term in an incorporated reference is inconsistent or contrary to the definition of that term provided herein, the definition of that term provided herein applies and the definition of that term in the reference does not apply.

In the last few years, it was found that treating the skin partially through what is called a fractionated treatment, reduced complications without affecting results greatly. Examples include ablative and non ablative fractionated laser treatment, and more recently radiofrequency fractionated treatment. Although laser and radiofrequency devices have been used to rejuvenate the skin, they still suffer from major drawbacks. One is a mechanical limitation where the size of a fractionated unit cannot be miniaturized to smaller units due to physical constraints. Another drawback is that such devices produce heating of the tissue and although heating may aid in rejuvenation, it is considered one of the main causes for complications. Yet another drawback is that only one or few related lasers can be used per device limiting the range of uses of that device. Since many medical laser devices typically involve heavy equipment, it can be difficult to maneuver these devices around the patient, which delays the treatment and makes reaching some areas difficult and cumbersome.

Yet another problem with lasers is their unpredictability in treating permanent hyperpigmentation disorders, such as Café au lait, congenital nevi and Becker's nevus. At times worse darkening has been seen due to bulk heating. Even when positive outcomes are seen, only about half of patients retain the results.

Due to these drawbacks, Medium and deep peels, such as phenol and trichloroacetic acid 35% and higher are falling out of favor as the treatment of choice for skin rejuvenation. Therefore there remains a considerable need for devices and methods that enhance a peel's rejuvenating potential without significant complications and with less downtime.

### SUMMARY OF THE INVENTION

The inventive subject matter provides apparatus, systems and methods in which chemical peels and other topical formulations can be applied to the skin in a standardized way, in which the amount of the peel, the depth of the peel, the area covered and other parameters, can be determined, and reliably given in either a fractionated or complete treatment to meet the goals for each patient. A fractionated treatment of the skin means that only part of the skin gets treated while surrounding areas are spared. The intact areas will help in the healing phase and will not greatly jeopardize the outcome of the treatment.

In another aspect of the inventive subject matter, a device includes a trigger activating a pump, which is continuous with a passageway that drains fluid from a reservoir to a nozzle. The trigger is operatively and pivotally coupled to the pump by a spring mechanism. The pump forces a predetermined volume of fluid to leave the nozzle with each stroke at the trigger. At the ends of the nozzle are a plurality of juxtaposed illuminators, they relay the distance to the operator by sending light beams to the area to be treated. A vacuum can be optionally used to ensure that the nozzle is dry after each treatment cycle and prevent any dripping from the nozzle to the skin. In another variation, the distance from the nozzle to the skin is measured by a stabilizer, one that can be removable and is anchored on the skin.

The nozzle can contain either a singular or a plurality of apertures through which droplets are expressed. The aperture/apertures are designed to allow only micron sized droplets to leave the nozzle to effect a fractionated to complete treatment. In one variation, the nozzle is detachable, with other nozzles mountable on the device, with different shapes and number of apertures.

The inventive subject matter further provides a method in which chemical peels can be reliably given to a patient. The device is held a predetermined distance from the skin before treatment is given. The distance is determined using any suitable distancing component, including for example an illuminator, a sonic distance measurer, and a physical stabilizer. Such distance is preferably pre-calculated to deliver the volume needed and end up in the density and intensity of treatment desired. In one variation, a concave shield that projects outwardly from nozzle margins can be used to limit spread of the droplets, and confine them to one area

The inventive subject matter also provides apparatus and methods by which the distance between the device and the skin can be precisely measured by the use of an illuminators or a stabilizer. This concept can be utilized in other devices like lasers and radiofrequency devices. Whenever a skin treatment has the potential for complications related to the incorrect positioning of the source of treatment to the skin, there will be an advantage of installing illuminators or stabilizers to protect against such complication. Therefore such treatment can be confidently standardized.

In another preferred embodiment, a device is held against the skin where the nozzle is held either right next to the skin or very close to it by a thin rim that surrounds and extends from the margins of the nozzle. Such embodiment will ensure proper localization of the droplets into their assigned fractionated imprints on the skin and limit unintentional spread and scatter of droplets.

The fluid used in treatments can contain any suitable compound, which can be present in a fluid or semi-fluid states. Chemical peeling are usually corrosive, and if not properly administered may end up in overdose and complications. Common examples of chemical peels include, phenol peels, trichloroacetic acid, glycolic, salicylic acid and Jessner's peels. Any topical treatment that is thought to cause irritation or complications is better given in a standardized manner, and/or as a fractionated application to gauge tolerance and monitor impending complications.

Yet another aspect of the inventive subject matter limits the unintended scatter, spread and coalescing of the droplets once they land on the skin, and ensures a fractionated treatment, additional agents can be optionally added into the peel solution prior to treatment. One example of such agents is Thickening agents. They increase the viscosity of the solution and therefore reduce the scatter and mobility of the droplets another variation, the thickening agent can be added not in the solution but on the skin itself evenly throughout treatment area before the treatment begins. Thus once the peel is sprayed into the skin they will resist merging into other droplets or scattering backwards.

The apparatus and methods in this invention can be used for cosmetic skin rejuvenation, where a general enhancement of the skin beauty is desired. This includes both facial and non facial areas. Positive outcomes usually sought include but are not limited to, unifying skin tone, reducing pigmentation, ameliorating wrinkles and laxity, increasing skin thickness and firmness and giving a youthful look. Other specific requests also may be targeted, such as lightening the skin, acne, acne scars, In short, all other known indication of chemical peels such as, seborrheic keratosis warts and milia, can be treated with this device with wider margins of safety and efficacy.

Various objects, features, aspects and advantages of the inventive subject matter will become more apparent from the following detailed description of preferred embodiments, along with the accompanying drawing figures in which like numerals represent like components.

### BRIEF DESCRIPTION OF THE DRAWINGS

The following detailed description, given by way of example and not intended to limit the present invention solely thereto, will best be appreciated in conjunction with the accompanying drawings, wherein like reference numerals denote like elements and parts, in which:

FIGS. 1 and 2 are side cross-sectional views of an exemplary device with the trigger both in relaxed (FIG. 1) and activated (FIG. 2) states. FIGS. 3 and 4 are similar but with the addition of vacuum pump.

FIGS. 5 and 6 are perspective views of two exemplary devices with two different illuminators while in operation.

FIG. 7 is a perspective view of an exemplary device having stabilizers.

FIG. 8 is a perspective view of an exemplary device with a single aperture while in operation.

FIG. 9 is a perspective view of an exemplary device with a shield.

FIG. 10 is a perspective view of an exemplary device with a multiple apertures while in operation.

FIGS. 11A through 11D are schematic diagrams of different nozzle variations.

FIG. 12 is a flow chart of the steps of giving a treatment according to one aspect of the inventive subject matter.

FIG. 13 is a flow chart of the steps involved in the device while in operation according to one aspect of the inventive subject matter.

FIG. 14 is a perspective view of an exemplary device

### DETAILED DESCRIPTION

The following discussion provides many example embodiments of the inventive subject matter. Although each embodiment represents a single combination of inventive elements, the inventive subject matter is considered to include all possible combinations of the disclosed elements. Thus if one embodiment comprises elements A, B, and C, and a second embodiment comprises elements B and D, then the inventive subject matter is also considered to include other remaining combinations of A, B, C, or D, even if not explicitly disclosed.

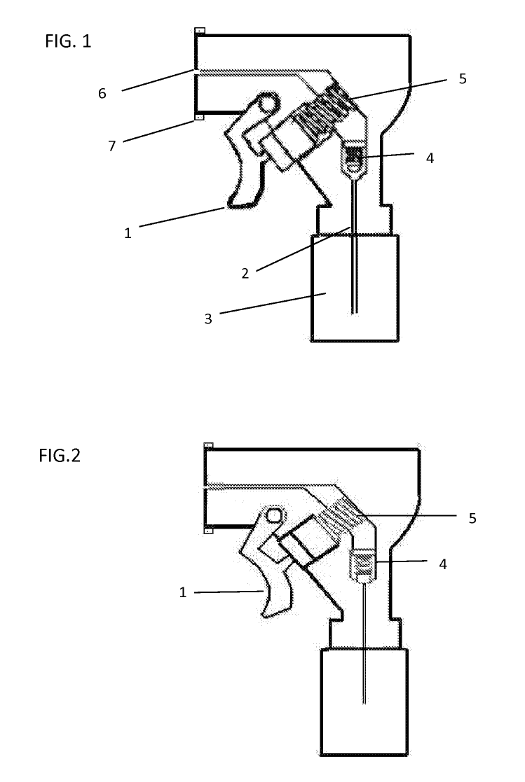

Referring to FIGS. 1, 2, 3 and 4, a device according to one embodiment includes a trigger 1, activating a pump 5 which is continuous with a passageway 2. This passageway drains fluid from a reservoir 3 to nozzle 6 when trigger is activated. The trigger is operatively and pivotally coupled to the pump by a spring mechanism. The pump forces a predetermined volume of fluid to leave the nozzle with each stroke at the trigger. At the upper and lower ends of the nozzle are two juxtaposed illuminators 7, they relay the distance to the operator by sending two light beams to the area to be treated. Finally a vacuum pump can be optionally used to ensure that the nozzle is dry after each treatment cycle and prevent any dripping from the nozzle to the skin.

The apparatus in one embodiment includes a vacuum pump 10 that when activated drains any lingering droplets from the nozzle from aperture 12 and into a storage area 11. Pressing the trigger once gives one treatment cycle. Droplets refer to the atomized solution particles that leave the nozzle before reaching the skin; they are small and can range in size from a few microns to 500 microns. These droplets are otherwise known as micronized fluid. The nozzle contains a singular or a plurality of apertures through which droplets appear. In one variation, the nozzle is detachable, with other nozzles mountable on the device, with different shapes and number of apertures.

In one variation, the pump 5 is of a conventional design and is designed to induce a negative pressure to draw fluid out. It includes a piston, housed inside a cylinder. Inside the cylinder, there is a spring. When the trigger is pulled back, this pushes the piston into the cylinder and the compresses the spring. When the trigger is released the piston is pushed back out of the cylinder and the spring is relaxed. As the piston is pushed in, this shrinks the area of the cylinder, forcing fluid out of the pump. When the piston goes back due to the springs recoil, it expands the cylinder area, sucking fluid into the pump. In one variation, to ensure a one way movement of fluid a valve 4 can be installed between the pump 5 and the reservoir 3. The valve comes in different designs, one of which is a rubber ball that rests inside a seal. The sides of the seal are configured so that the ball won't fall out. Other variations in pump design known to those skilled in the art such as those incorporating electrical and automated pumps can also be used.

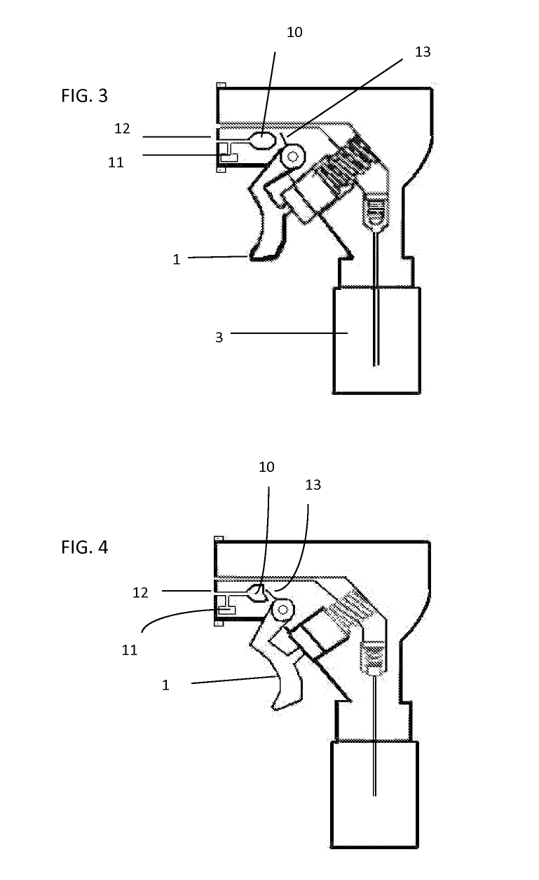

During operation, as shown in FIGS. 1 and 2, the device is held a predetermined distance from the skin aided by the illuminator, such distance is pre-calculated to deliver the volume needed and end up in the density and intensity of treatment desired. The density refers to how far the droplets are from each other once they land on the skin, and the intensity refers to the size of the droplets. If the device is held too close or too far, the same amount of volume will end up in a denser or less dense treatment. FIGS. 3 and 4 describe a similar treatment but with the use of a vacuum pump. An arm 13 is connected to trigger 1 and moves with it. When the trigger is actuated, arm 13 moves towards vacuum pump 10. When vacuum pump gets squeezed it generates a negative pressure that draws any leftover fluid from nozzle 6 through aperture 12. The excess fluid can be stored in storage area 11, which in one variation is in connected to the reservoir 3 to be recycled for subsequent treatments. Electrical signals can be used to link the vacuum pump to the trigger device. The timing of the vacuum action is critical and should follow each treatment cycle and not coincide with it.

Referring to FIG. 5 the distance from the nozzle to the skin can be measured by an illuminator 7. Here, two illuminators are juxtaposed on each side of the nozzle. The light source is given at angle in such a way that the two lights converge to meet at a predetermined distance from the skin, one of which has been chosen for the treatment. holding the apparatus too close or too far from the desired distance will produce two laser imprints alarming the operator to adjust the distance accordingly and try to align the nozzles surface parallel to the skin. The two dotted lines in FIG. 5 correspond to imaginary false closer and further positions away from the desired correct position where the treatment should be given. The source of light used can be of different types. Preferably a Stronger illumination such is with laser type illumination can be used. An example is a diode source lasers. Other examples include but are not limited to neon, helium, ND YAG lasers. If needed a weaker illumination such as that with LED light is used

In another variation of the last embodiment (not shown in figures), the illuminators can be increased to four each at one side of the nozzle. This will correspond to four laser imprints on the skin, and only when all of the four laser imprints are focused into one point that the operator is at the right distance and can start treatment. By adding two more illuminators, the operator can adjust the device in two planes, adding to the precision of the treatment. Different colors can be used for each pair at each plane to aid the operator to move the device in the right direction.

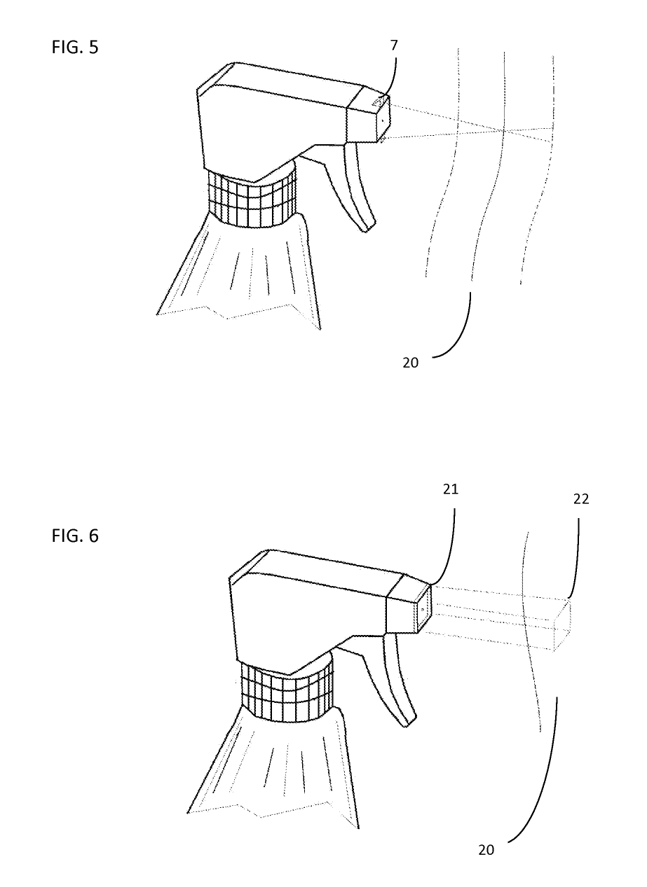

Referring to FIG. 6, in another variation, and to measure the distance from the skin the device can be equipped with a square like beam source 21, disposed at the margins of the nozzle. This beam will give a shape onto the skin 22 corresponding to the nozzle's shape and will constitute the area to be treated. Only when the lines drawn on the skin is sharp, the desired distance has been reached and treatment can be given. Deviating from that desired distance will produce fuzzier lines on the skin and alarms the operator to readjust the distance accordingly. The source of the beam can be a laser diode or other examples like the ones given previously.

In another variation as shown in FIG. 7 the distance between the nozzle and skin can be determined by use of a physical stabilizer by which the device can be rested on the skin. The stabilizer is elongated and extends from the nozzle outwardly. The stabilizer can be of any suitable shape, and is usually made of plastic. For the purpose of delivering corrosive peels to the full face the stabilizer is preferably limited to one corner of the treatment area (figure shows two stabilizers for exemplary purposes) with only a few resting projections at its base 25 or none 26. This will minimize imprinting a peel from a previously treated area to a new area, doubling a treatment cycle unintentionally. Another way of minimizing this doubling is to treat the skin systematically from one area to an adjacent area at the same direction so that the stabilizer never falls on a previously treated d area. The length of the stabilizer is the predetermined distance for the treatment, which varies according to the area treated and the speed of the ejected droplets, but in most indications a distance between 2 cm and 4 cm, inclusive, is suitable.

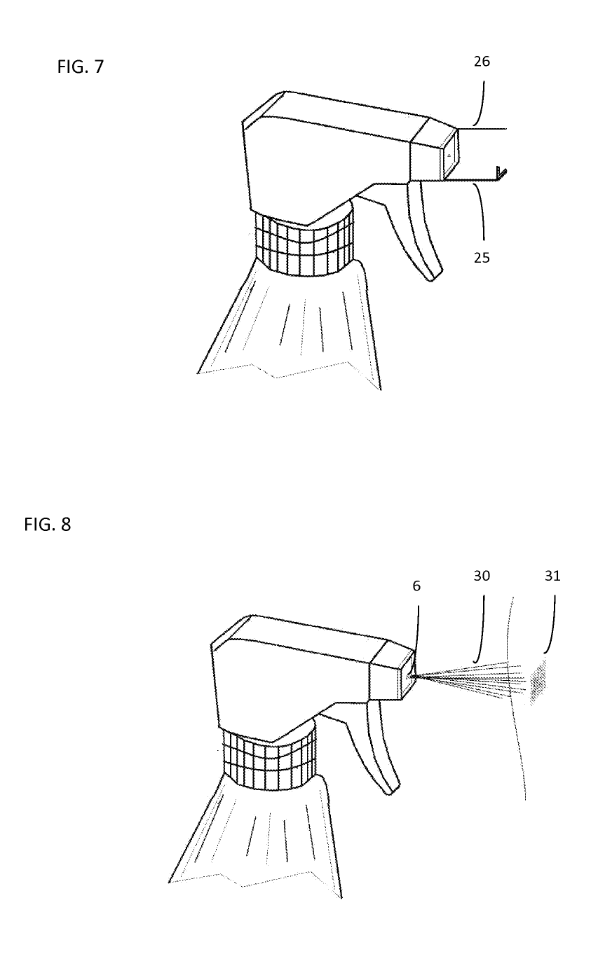

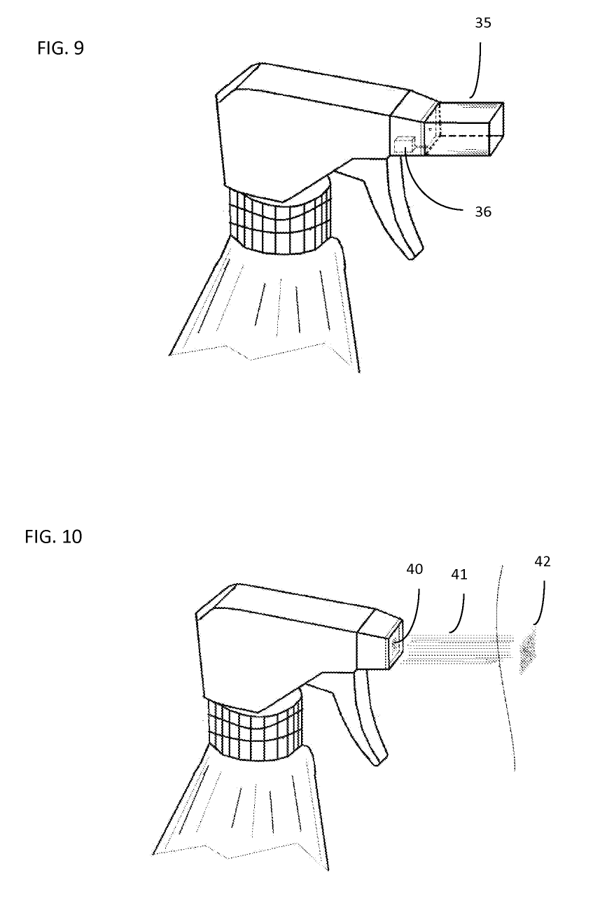

Referring to FIGS. 8, 9 and 10 describes what a treatment cycle would look like. Optionally, a shield 35 that projects outwardly from nozzle margins and is hollow from within can be used to limit spread of the droplets and confine it to one area as shown in FIG. 9. This will further limit the spreading of the droplets which might be breathed by the patient or operator. A face mask can be used as an extra safety measure during the procedure. It can also be combined with a vacuum unit 36 to ensure that leftover droplets will not spell inadvertently into skin. The way the treatment cycle can be delivered in many ways. One way as show in FIG. 8 when only one aperture is designed at the nozzle and this causes the droplets to concentrate at the centre and fade towards the periphery. The stream of droplets may diverge as they leave the nozzle. Such fading might be desirable when subsequent treatments are given adjacent, and will help in a better and safer overlap pattern and reduce cutoff lines at the outside boundaries of the treatment area. Another way is to have a plurality of apertures at the nozzle as shown in FIG. 10 with the density of treatment being homogenous. This will mean that droplets travel parallel to each other and all parts of the skin treated at that area gets the same amount of volume. Overlap should preferably be reduced to no more than 40-30% of coverage depending on the nozzle used.

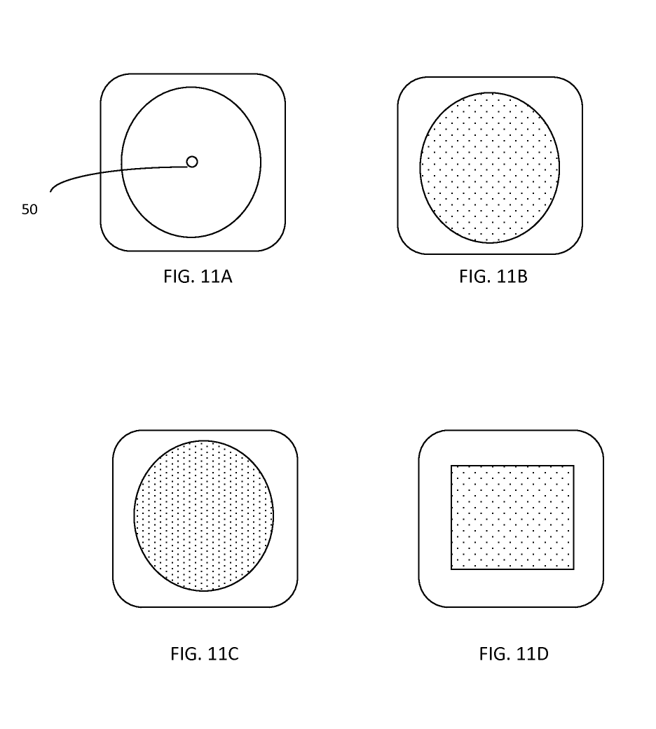

Now referring to FIGS. 11A through D. since there is a range of skin types to be treated and different intensities of treatment tailored to each patient's expectation, there are many possible variations of nozzle shapes to be considered. The aperture 50 in the nozzle can be singular and central as shown in FIG. 11A giving a fading type of fractionated treatment with a more concentrated treatment at the middle. In another variation, any number of apertures can be used to deliver a more uniform type of fractionation. This uniform fractionation can be with less density as shown in FIG. 11B or a denser treatment as shown in FIG. 11C. The shape of the treatment can also be varied if the nozzles shape is changed to square to other shapes such as square as shown in FIG. 11D. The size and number of the apertures and the distance from one another can be varied according to the density and intensity of treatment chosen, they can range from a few microns to about 500 microns in size, and be anywhere from a few microns to about 500 microns from one another. The number of apertures depends mainly on the area treated, with larger areas necessitating more apertures

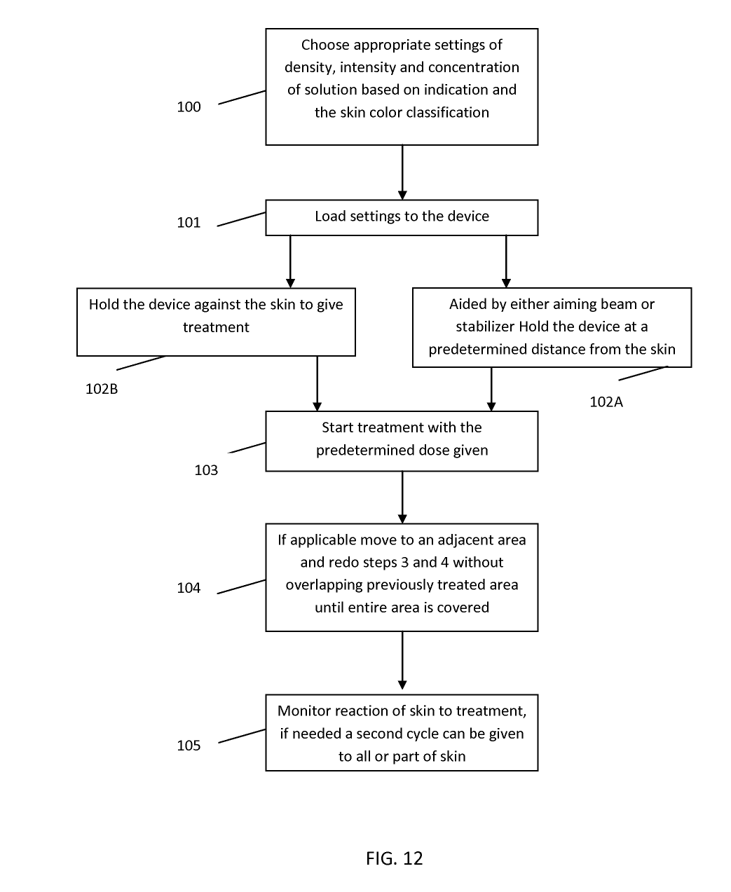

FIG. 12 describes the steps needed for a fractionated chemical peel treatment. It is preferable for practical reasons that a prefixed distance is used to deliver a predetermined fractionated treatment for all range of possible treatments. The fractionated treatment depends mainly on the intensity and density of the treatment. The density and intensity depends mainly on the number and size of apertures in the nozzle along with the volume given. The operator chooses the type of peel, load it in the reservoir area and uses the right nozzle with its unique number of apertures and unique distance between these apertures (there could be a kit of detachable nozzles as will be discussed later) giving a specific density and intensity. The treatment starts with the device held at a predetermined fixed distance from the patient skin, aided by an illuminator or a stabilizer 102A. Once the right distance has been reached the operator presses the trigger once giving one treatment cycle 103. Then if applicable, the operator moves to an adjacent area and repeats the same steps until all areas are treated. The operator should watch for the reaction of the skin to the peeling and may choose to give more coats if needed. With each treatment, as long as the distance is constant and only one treatment cycle is given per area with minimal or no overlapping, a constant and even peeling of the skin will be produced. The same methods can be used by another embodiment that is held against the skin but with step 102B instead of 102A as will be explained later.

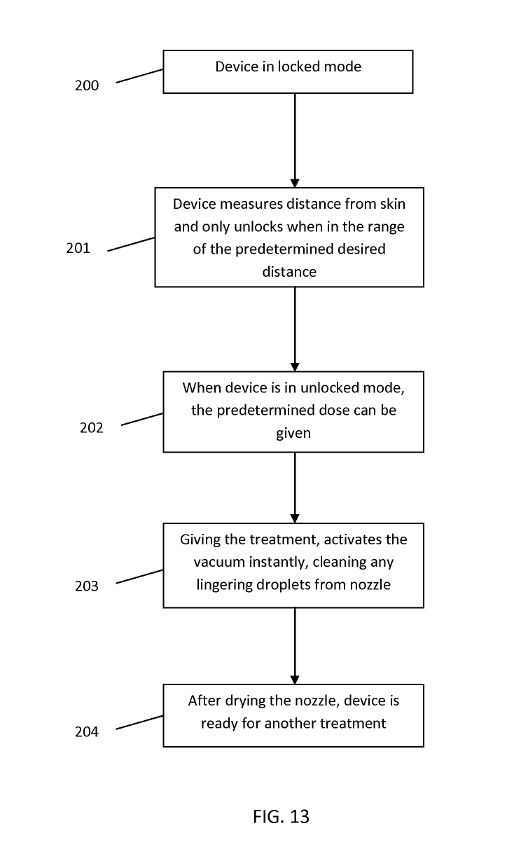

Referring to FIG. 13 a method of operation of the device is described. Whenever a treatment cycle is given, a vacuum unit is activated 203, draining any excess fluid from nozzle and preparing the device for the next cycle. Steps 200 and 201 are optional, it further increase precision of the device by locking the device if not at the range of the proper distance and can be incorporated only in specific devices as will be discussed later.

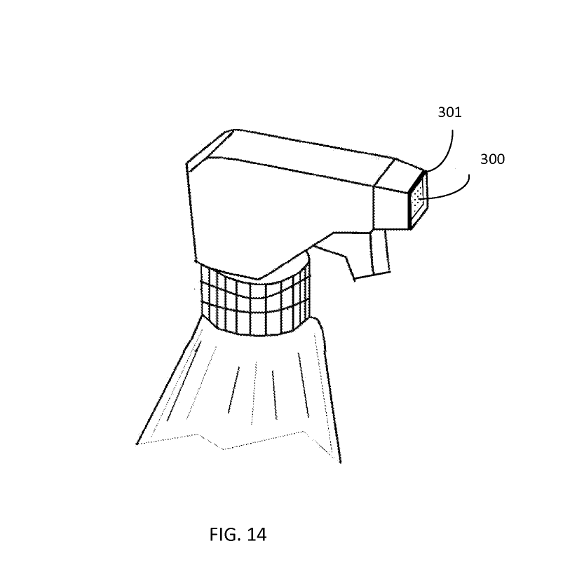

Referring to FIG. 14 in another preferred embodiment, the device is similar to the previous embodiments, except that instead of using it away from the skin to spray droplets at a predetermined distance, it is held against the skin. The nozzle 300 should be held either right next to the skin or very close to it (approximately 1-3 mm away) by a thin rim 301 that surrounds and extends from the margins of the nozzle 300. All variations of nozzles shown in FIGS. 11A to 11D can be used in this embodiment. With close contact of apertures with the skin, this will ensure proper localization of the droplets into their assigned fractionated imprints on the skin and limit unintentional spread and scatter of droplets. Using this embodiment is similar to steps shown in FIGS. 12 and 13 except that they will be placed against the skin. Step 102B is used instead of step 102A. As the case with previous embodiments, vacuum pump can be used between treatment cycles to prevent inadvertent spillage of lingering droplets on the nozzle.

As the case with laser and radiofrequency device, there are generally two ways to apply the treatment to the skin. The first way that has been described so far is the static mode, where only one area is treated per treatment cycle. The operator adjusts the location of the device to an area adjacent the treated area and gives another treatment cycle until the whole area has been treated. Another optional way is known as dynamic mode. In this mode the device once activated keeps spraying the droplets continuously as the operator keeps moving to other areas until it is switched off. The dynamic mode will reduce the time of the procedure considerably, but it is less precise than the static mode. It is better suited for less intense treatments where minor deviations would not cause major complications. The speed of the moving hand in the dynamic mode is crucial as giving faster speeds will end up in less density and slower than usual speed will give a higher than desired density.

The distance between the nozzle and the skin is a parameter that is preferably fixed for the purpose of standardizing the treatment as is the case with laser and radiofrequency devices. By fixing this parameter, the density and intensity of treatment are the main parameters to be adjusted according to factors related to the patient and treatment goals. Limiting this distance will limit drifting of the droplets as they travel from the nozzle to the skin. Longer distances can be used when a larger nozzle is used to treat all of the face in one treatment cycle. In such device the nozzle is curved to cover all parts of the face. This distance, of course, can be established using an illuminator or a stabilizer as described above.

The fluid or liquid used in reservoir 3 can contain any Compound present in a fluid or semi-fluid states. Chemical peeling are usually corrosive and if not properly administered may end up in overdose and complications. The procedure would be safer if such treatment can be standardized in one aspect and if the skin can be treated fractionally. A fractionated treatment of the skin means that only part of the skin gets treated while surrounding areas are spared. The intact areas will help in the healing phase and will not greatly jeopardize the outcome of the treatment. Lasers, radiofrequency and mechanical needles have been used to treat the skin fractionally, and proved to be safer than more traditional skin treatment. The intensity and density of a fractionated treatment is usually tweaked according to the patient's skin type, with darker skin more prone to complications and therefore lighter settings of treatment are used. Other factors to be considered include the location of treatment with non facial areas prone to complications. Another important factor is the patient expectation with some welling to go for a more intense treatment and longer recovery than having a series of milder treatments spaced apart. The choice for the peel type and its concentration depends on the same factors listed above.

Different shapes and sizes of the nozzle can be used to administer different treatments to reach different goals and this will be discussed in details later. To standardize a treatment cycle of a chemical peel, one that can be reproducible and predictable each time the trigger is used; three parameters must be addressed before starting treatment, 1-distance from nozzle to skin to be treated, 2- nozzle's shape and number of apertures it has and 3- volume given per treatment cycle. The trigger 1 will always spray the same amount of solution per unit treatment area. The volume is dependent on the sizes of the passageway2, the pump 5 and how much the trigger moves. The volume is calculated in concordance with the corresponding density and intensity of treatment, and is advantageously chosen according the size of the treatment area. The density of treatment refers to the percentage of treated area, which in most conditions range from 10-90% treatment coverage. Once they land, the distance of one droplet to another is another way of defining density, with smaller distances between droplets, equating higher density of treatment. 100% coverage area is referred to a complete treatment is can be given in situations like when superficial and mild peels are given. Even in complete treatment, using any embodiment described in this invention would lead to a standardized and reproducible treatment. Preferably this distance, is substantially constant all over the treatment area to harmonize the results. In other situations, a variable distance can be utilized to simulate the haphazard arrangement of skin tone present in normal skin or to taper the density at the periphery to allow for a safer overlap. Such pattern is best utilized by a single aperture.

The intensity of treatment refers to the size of droplets, with larger droplets having deeper penetration and more intense treatment. The size of droplets is mainly determined by the nozzle's shape 6 and size of apertures they hold. Larger apertures will produce larger droplets. Droplet size is also affected by the volume given in unit time and the speed by which they are given. Although higher speeds produce more uniform fractionation and less gravity induced deviation they may increase backward scatter of the droplets. Choosing the right density and intensity is carefully gauged to the patient's skin type and aim of treatment. for instance, a treatment for general rejuvenation with a minimal downtime that is given to even skin tone and give a modest peeling of the skin in a white skin (skin type 1 or 2) can be performed with a trichloroacetic acid (30 percent concentration) with a density around 70-80%. However if the same patient desires amelioration of wrinkles and or reducing laxity of the skin then a higher density or intensity of treatment with even a higher concentration of trichloroacetic acid solution, should be given to reach these goals.

Unless the context dictates the contrary, all ranges set forth herein should be interpreted as being inclusive of their endpoints and open-ended ranges should be interpreted to include commercially practical values. Similarly, all lists of values should be considered as inclusive of intermediate values unless the context indicates the contrary.

The nozzle 6 is configured in a way that it only sprays carefully sized droplets and a predetermined volume to the skin in a way that ensures a fractionated treatment. The device is not intended to wet the area but to deliver a fractionated treatment. Preferably droplet size leaving the nozzle can range from about 5 to 500 microns, and the volume given per treatment cycle can range from 0.1 ml to 1 ml for a 1 cm² area, depending on the patient's tolerance, his or her skin type, and the condition to be treated if applicable. However smaller droplets (less than 10 microns) can rarely be used in selected conditions such as when there a high probability for complication with very dark skin.

Other ways to gauge the distance known to those skilled in the art that can be included, include ultrasound driven mechanisms that measure the distance by sending waves to the skin, which rebound back and are used in calculating the distance. The distance can be relayed to the operator by a screen or a beeping sound that for instance stops beeping when in the range of the desired distance. The same method can also be accomplished by laser technologies. With such technologies it is possible to program the device to be in a locked mode if not in the range of the right distance from the skin by using a software program that connects different parts of the device.

The advantages of a free treatment, one that doesn't involve touching the skin (as the case with FIGS. 5 and 6) are many. Firstly, this will make reaching difficult areas easier like around the eyes and the nose. Secondly, the treatment field is entirely visible for the operator for assessment as he continues treatment with a better overlapping pattern and a faster treatment. unlike laser and radiofrequency devices where changing the distance of treatment greatly reduces the effect of the treatment, the peel is more forgiving with minimal unintentional changes in distance, since the droplets would eventually fall on the skin. This is especially true with one variation of the nozzle shown in FIG. 10 where the droplets come out of the nozzle in parallel fashion from many apertures.

Since the droplets are sprayed to be individually placed on the skin creating a fractionated treatment, and to ensure such placement, additional agents can be optionally added into the peel solution prior to treatment to limit the movement and scatter of the droplets once they land on the skin. This is especially useful when higher densities are desired (and therefore droplets are closer to each other) or when larger droplets are used. Larger droplets can migrate by force of gravity into neighboring droplets coalescing into larger droplets and turning the fractionated treatment into a complete one. A non-limiting example of these agents is Thickening agents. They increase the viscosity of the solution and therefore reduce the scatter and mobility of the droplets once they touch the skin. By using such agents the speed by which the droplets travel to the skin can be increased without increasing the rate of scatter. In another variation, the thickening agent can be added not in the solution but on the skin itself evenly throughout treatment area before the treatment begins. Thus once the peel is sprayed into the skin they will resist merging into other droplets or scattering backwards.

Thickening agents include many agents. Examples include but are not limited to petroleum jelly, polyethylene glycol, synthetic, polyacrylic acid, vegetable gums., petroleum jelly, waxes, Clays (Aluminum Magnesium Silicate, Bentonite and Hectorite) and synthetic agents (Carbomer, Colloidal silicon dioxide) Biphenyl alcohol, Bis-diglyceryl polyacyladipate. Behenic acid Crosslinked Vinyl Ether Maleic Anhydride Copolymers, Norbak and Vistik and tragacanth. Other non-limiting examples of carboxylic acid polymers include acrylic acid, methacrylic acid, ethacrylic acid, pentaerythritol. Other examples of Polyacrylamide Polymers include acrylamide, methacrylamide, N-methacrylamide, N-methylmethacrylamide, N,N-dimethylmethacrylamide, N-isopropylacrylamide, N-isopropylmethacrylamide, and N,N-dimethylacrylamide.

Non-limiting examples of polysaccharide gelling agents include those selected from the group consisting of cellulose, carboxymethyl hydroxyethylcellulose, cellulose acetate propionate carboxylate, hydroxyethylcellulose, hydroxyethyl ethylcellulose, hydroxypropylcellulose, hydroxypropyl methylcellulose, methyl hydroxyethylcellulose, Hydroxypropyl Methyl Cellulose, microcrystalline cellulose, sodium cellulose sulfate, carboxymethyl mungbean starch, carbomer and mixtures thereof.

Natural thickening gents can be used too. Non-limiting examples of these agents include materials selected from the group consisting of acacia, agar, align, alginic acid, ammonium alginate, Sodium Alginate amylopectin, calcium alginate, calcium carrageenan, camitine, carrageenan, dextrin, gelatin, gellan gum, guar gum, Gum Tragacanth, Gum Acacia guar hydroxypropyltrimonium chloride, hectorite, hyaluroinic acid, hydrated silica, hydroxypropyl chitosan, hydroxypropyl guar, karaya gum, kelp, locust bean gum, natto gum, potassium alginate, potassium carrageenan, propylene glycol alginate, sclerotium gum, sodium carboxymethyl dextran, sodium carrageenan, tragacanth gum, xanthan gum, and mixtures of them.

The concentration of the thickening agent varies according to the type of peel used and the density of treatment. Thickening agents usually have a concentration that ranges from 0.5% to 5%, however concentration not included in this range has been used with comparable results.

Other agents that might be used to increase the viscosity of the solution and limit spread are binding and gelling agents. Kollidon is an example of a binding agent, whereas nonl-imiting examples of a gelling agent include, agar, calcium alginate, carageenan, and pectins.

Yet, other agents that increase the viscosity of a solution and adherence include binders. In certain situation where extra viscosity needed these agents can be used. Non-limiting examples of binders include Saccharides and their derivatives like Disaccharides such as sucrose, lactose; Polysaccharides and their derivatives such as cellulose or modified cellulose such as microcrystalline cellulose and cellulose ethers such as hydroxypropyl cellulose (HPC); Sugar alcohols such as xylitol, sorbitol or maltitol; Protein such as gelatin, Synthetic polymers such as polyvinylpyrrolidone (PVP), polyethylene glycol and Gum Arabic

The choice of compounds used in the device can be any chemical peels. This is contrary to laser and radiofrequency devices where the range is severely limited. Common examples of chemical peels include, phenol peels, trichloroacetic acid, glycolic, salicylic acid and Jessner's peels. Other non-limiting examples include lactic acid, puruvic acid, other alpha hydroxyl acids, other beta hydroxyl acids, malic acid, citric acid and Resorcinol.

The choice for the peel's concentration should be decided after considering the patient's skin type, with darker skin prone to complications and having less concentrated peels. Other considerations include the aim of the treatment, the patient tolerance and expectation, the part of the body to be treated with non facial areas tolerating more concentrated peels. For example a trichloroacetic acid at a concentration of 30% (concentrations of peels are by weight per unit volume) can be used to treat hyperpigmentation in very light white skin whereas the same condition in a very dark skin should be treated with concentration around 15%. Using any device described above in the preferred embodiments, the concentration of the peels used can even go higher since the skin has been treated partially. Traditionally, deep peels done with agents like phenol and trichloroacetic acid (in higher concentrations). Significant rejuvenation can be produced best with deep peels. Using a deep peel with any one of the embodiments or methods described in this disclosure are expected to result in a deeper and safer peel that will produce better results. This will translate into a more youthful face with less downtime and less chance for complications.

The reservoir is where any type of peel is stored prior to treatment. In one variation this reservoir area is disconnectable, so that the operator need not to transfer the peel from its container to the reservoir area but rather is ready for use once it has been connected to the device. It houses the passageway2 that drains the fluid. Likewise, in one variation the nozzle assembly 6 can be disconnectable from the device, where different nozzle shapes and densities can be mounted on the device for different treatment indications. They may come with a singular aperture to a plurality of them and with different size of each aperture and different distance from one aperture to another. Furthermore, different disconnectable reservoir compartments (corresponding to different peel concentrations) and different disconnectable nozzles (corresponding to different density and intensity of treatment) can be provided in a one kit, to cover all ranges of skin types and treatment indications. Therefore the operator installs the reservoir and nozzle each time he performs a treatment on a different patient or a different area on the same patient, reusing the device for all treatment options. Furthermore the volume of solution given per treatment cycle can be adjustable in one variation and can be changed to the other parameters chosen such as treatment density, intensity and area of treatment. Adjustment of the volume can be done in many ways known to those skilled in the art, one of which is extending the range of the movement of the trigger to be able to withdraw smaller to higher volumes of solution. To inform the operator marks can be printed on the device corresponding to the range of movement used and therefore the volume given.

In a variation, the aforementioned mechanisms, methods and embodiments can be programmed with a software computer that has all the preferred settings of treatment density, intensity for different unit areas in concordance with the pre-calculated distance and volume given per treatment cycle. It is understood that in such device, electrical units are installed to connect various parts of the device and motor units activated by electrical switches are used to draw the solution from the first reservoir area rather than using a manual trigger. Other actions such as the speed of the droplets can also be saved in the software and controlled by the corresponding units. Furthermore the speed of the treatment in the dynamic mode and the mechanism by which activating the vacuum unit can also be controlled by the software and corresponding units. This will increase precision and simplify the procedure. Furthermore, as the case with laser and radiofrequency devices known in the prior art, the nozzle shape and number of apertures can be varied without disconnecting the nozzle from the device. Screens can be used to display and choose appropriate settings and sounds are used to alarm the operator of events like, distance from skin, starting and ending treatment.

While chemical peels, are described in detail above, it is understood that any topical solution can also be used with any one of the embodiments described or methods in this disclosure. This is especially true for corrosive agents, and exfoliants. These agents tend to irritate the skin. Non-limiting examples include 5-Fluorouracil, immiquimod, or agents used in photodynamic therapy such as aminolevulinic acid, methyl aminolevulinate or levulinic acid. In short any topical treatment that is thought to cause irritation or complications is better given in a standardized manner and/or as a fractionated application to gauge tolerance and monitor impending complications.

While solutions are mentioned in detail above, it is understood that any compound present in a fluid or semi fluid state can be used in conjunction with the inventive concepts herein. Non-limiting examples of such vehicles include solutions, lotions, and ointments. The choice of the type of vehicle depends on the viscosity of the compound and whether thickening agents have been used.

Illuminator and stabilizer can be utilized in other devices known in the prior art. Laser and radiofrequency devices are non-limiting examples. Whenever a skin treatment has the potential for complications related to the incorrect positioning of the source of treatment to the skin, there will be an advantage of installing illuminators or stabilizers to protect against such complication. Therefore such treatment can be confidently standardized.

Other devices that employ mechanisms used to atomize fluid into smaller droplets can be used instead of what has been described above. Examples include but are not limited to pneumonic sprayers, or humidifiers devices such as impeller, steam, ultrasonic or Wick/Evaporative System can all be used. However it is important that fluid, instead of just gaseous material, be sprayed into the skin.

The embodiments described in this disclosure are mainly intended for skin treatment, mainly for cosmetic skin and hair rejuvenation, where a general enhancement of the skin beauty is desired. This includes both facial and non facial areas. Positive outcomes usually sought include but are not limited to, unifying skin tone, reducing pigmentation, ameliorating wrinkles and laxity, increasing skin thickness and firmness and giving a youthful look. Other specific requests also may be targeted, such as lightening the skin (otherwise known as bleaching), treating melasma, postinflammatory hyperpigmentation, freckling and pigmentary problems in general. Scars are cosmetically unacceptable, and conditions like acne scars, striae and other scars can all be treated. The wade range of indications could only be possible with versatility of settings (density, intensity, speed of droplets etc.) that can be utilized in this device. In short, all other known indication of chemical peels (such as, Seborrheic keratosis warts and milia) can be treated with this device with wider margins of safety and efficacy.

Another indication for treatment is permanent hyperpigmentation disorders that may or may not arise at birth and for which there is no cure. The embodiments and methods desrcribed in this disclosure can be used to permanently, ameliorate or cure the condition. One of the complications of chemical peels is post inflammatory hypopigmentation, which can be utilized to lighten the skin. This complication arises usually with higher concentrations of peels and deeper peels. Since these conditions are permanent, it would be suitable to lighten the skin permanently with a deep fractionated treatment aimed at destroying melanocytes fractionally. Similar to pixilating a dark colored sheet into lighter hues by introducing lighter colors at equidistant points to produce a fractionated pattern, the device is used to introduce the peel fractionally, to intentionally cause post inflammatory hypopigmentation and ultimately lighten the hyperpigmentation. This can be done in several sessions, to ensure that one treatment doesn't overtreat the area, inadvertently becoming lighter than surrounding skin. Non limiting examples of diseases that can be treated with this include congenital diseases such as congenital melanocytic nevus, and café au lait spots. A non-limiting example of a condition that arises later in life is Becker's nevus.

Thus, specific compositions and methods of this invention have been disclosed. It should be apparent, however, to those skilled in the art that many more modifications besides those already described are possible without departing from the inventive concepts herein. The inventive subject matter, therefore, is not to be restricted except in the spirit of the disclosure. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

As used herein, and unless the context dictates otherwise, the term “coupled to” is intended to include both direct coupling (in which two elements that are coupled to each other contact each other) and indirect coupling (in which at least one additional element is located between the two elements). Therefore, the terms “coupled to” and “coupled with” are used synonymously

What is claimed is:

1. An apparatus for treatment of skin, comprising:
a nozzle having an aperture;
a first reservoir containing a first treatment fluid;
a passageway delivering the fluid from the reservoir to the nozzle;
a pump operatively coupled to the passageway to propel the fluid from the reservoir towards the nozzle;
a trigger operatively coupled to the pump to move a predetermined volume of the fluid; and
a distancing component that assists in maintaining a predetermined non-zero distance between the nozzle and the skin.

2. The apparatus of claim 1, wherein the distancing component comprises a plurality of illuminators configured to converge light beams at the predetermined distance.

3. The apparatus of claim 1, wherein the distancing component comprises an illuminator that focuses an image at the predetermined distance.

4. The apparatus of claim 1, wherein the distancing component comprises a diode laser

5. The apparatus of claim 1, further comprising a vacuum disposed to remove excess droplets from the nozzle.

6. The apparatus of claim 1, wherein the aperture has a size that ranges from about 5 microns to 500 microns, inclusive.

7. The apparatus of claim 1, wherein the predetermined distance ranges from 2 cm to 4 cm, inclusive.

8. The apparatus of claim 1, wherein the distancing component comprises a stabilizer physically projecting outwardly from the apparatus by at least 1 cm.

9. The apparatus of claim 1 further comprising a shield projecting outwardly from the nozzle.

10. The apparatus of claim 1, wherein the first treatment fluid includes at least one member of the group consisting of: phenol, trichloroacetic acid, glycolic acid, salicylic acid, Jessner's peel, aminolevulinic acid and methyl aminolevulinate.

11. The apparatus of claim 1, wherein the nozzle is user-replaceable with different sized nozzles.

12. The apparatus of claim 1, further comprising a second reservoir having a second treatment fluid different from the first treatment fluid, and the first reservoir is user-replaceable with the second reservoir.

13. A system for treatment of skin, comprising an apparatus according to claim 1, and an electronic system that controls at least one of droplet size, predetermined volume, and treatment density.

14. A method for treatment of a skin, comprising:
providing a sprayer having (a) a reservoir, and (b) a nozzle, configured to produce micron-sized droplets;
adding a treatment fluid to the reservoir;
holding the sprayer a predetermined distance from the treatment area;
spraying a predetermined volume of the fluid onto the treatment area to produce a fractionated treatment.

15. The method of claim 14, further comprising the step of using an illuminator to assist in holding the sprayer a predetermined distance from the treatment area.

16. The method of claim 14, further comprising the step of using a physical stabilizer to assist in holding the sprayer a predetermined distance from the treatment area.

17. The method of claim 14, further comprising adding a thickening agent to the skin prior to the step of spraying a predetermined volume of the fluid onto the treatment area.

18. The method of claim 14, further comprising adding a thickening agent to the treatment fluid prior to the step of adding a treatment fluid to the reservoir.

19. The method of claim 14, further comprising activating a vacuum to dry the nozzle.

20. The method of claim 14, wherein the fluid comprises one of the following: phenol, trichloroacetic acid, glycolic acid, salicylic acid, Jessner's peel, aminolevulinic acid, methyl aminolevulinate, levulinic acid or a combination thereof.

21. The method of claim 14, wherein the predetermined distance is between 2 cm and 4 cm, inclusive.

22. An apparatus for spraying a liquid onto the skin, comprising:
a nozzle having an aperture to be held tight against the skin;
a reservoir adapted to contain the liquid;
a passageway delivering the liquid from the reservoir to the nozzle;
a pump operatively coupled to the passageway to propel the liquid from the reservoir towards the nozzle;
a trigger operatively coupled to the pump to move a predetermined volume of the liquid; and
wherein the nozzle is configured to deliver a micronized fractionated treatment.

23. A method for treating pigmentation on the skin comprising the steps of
providing a sprayer having (a) a reservoir, and (b) at least first and second interchangeable nozzles, each of which produces micron-sized droplets;
selecting among a plurality of treatment settings for the sprayer;
using a distancing apparatus to hold the sprayer a predetermined distance from the skin; and
spraying the solution into the skin in a fractionated way.
